(19) United States
(12) Patent Application Publication
(10) Pub. No.: US 2008/0097519 A1
(43) Pub. Date: Apr. 24, 2008

(54) MEDICAL DEVICE EXTRACTION TOOL

(75) Inventors: Joseph L. Calderon, Porter Ranch, CA (US); Joseph H. Schulman, Santa Clarita, CA (US); Edward Hillery, Valencia, CA (US)

Correspondence Address: ALFRED E. MANN FOUNDATION FOR;SCIENTIFIC RESEARCH, PO BOX 905, SANTA CLARITA, CA, 91380, US

(73) Assignee: ALFRED E. MANN FOUNDATION FOR SCIENTIFIC RESEARCH, Santa Clarita, CA (US)

(21) Appl. No.: 11/551,433

(22) Filed: Oct. 20, 2006

(51) Int. Cl. A61B 17/00

(52) U.S. Cl. 606/205

(57) ABSTRACT

A medical instrument, particularly a surgical instrument with a displaceable push/pull activation tube arranged on the proximal end of a hand manipulator, that also contains an inner tube for retaining a fixed jaw, for activating remote tool parts on the distal end, in which a force-limiting device limits the transmission of force to the remote tool parts from the hand manipulator via an extendable coil spring. The medical instrument optionally features a fiber scope to enable optical viewing to locate the implanted device for removal as well as a saline flush capability. The instrument has jaws that are cylindrical in shape to facilitate grasping an implanted cylindrical device.

### BACKGROUND OF THE INVENTION

The invention relates to a medical instrument, particularly a surgical instrument with a displaceable push/pull rod arranged on the proximal end of a hand manipulator for activating remote tool parts on the distal end, wherein a force-limiting device is envisaged for limiting the transmission of force from the hand manipulator onto the remote tool parts via the push/pull rod.

This kind of medical instrument can be a removal tool for microstimulators or microsensors, as well as a needle holder, a gripping, holding or preparation tool, scissors or other instrument, in which the push/pull rod can be moved back and forth using manual force via the hand manipulator, in order to move, i.e. to open and close, the remote tool parts which are predominantly open-ended tool parts.

The specific microstimulators of interest to the instant application are small, approximately 6 mm diameter by 30 mm long cylinders, implantable electrical devices that pass a small signal to living tissue in order to elicit a response from a nerve or muscle. Microsensors are similar electrical devices except that they detect electrical and other signals that are generated by living tissue. The term microstimulator is intended to apply equally to both microstimulators and microsensors. See for example, U.S. Pat. Nos. 5,193,539; 5,193,540; 5,324,316; 5,405,367; 6,175,764; 6,181,965; 6,185,452; 6,185,455; 6,208,894; 6,214,032; and 6,315,721, which are incorporated in their entirety by reference. These microstimulators are particularly advantageous because they can be implanted by injection. Microstimulators, as exemplified by the BION® of Advanced Bionics Corporation, are typically elongated devices with metallic electrodes at each end that deliver electrical current to the immediately surrounding living tissues.

These medical instruments available in various embodiment configurations have a long hollow cylindrical shaft, onto the distal end of which the remote tool parts are arranged. See for example U.S. Pat. Nos. 6,589,259 and 6,840,932, which are incorporated by reference in their entirety. The hand manipulator with a rigid handle element and a swiveling handle element is arranged on the proximal end of the shaft. To activate the remote tool parts via the hand manipulator, the remote tool parts and the swiveling handle element of the hand manipulator are coupled via the push/pull rod which is located in the hollow cylindrical shaft. In this way it is possible to open and close the remote tool parts by counter-adjusting.

These types of medical instruments are often used during minimally invasive surgery. Due to the miniaturization of the instruments required for minimally invasive surgery, the instruments are more sensitive to pressure since, due to miniaturization, the individual components can only absorb marginal forces, which for example are brought about by hand pressure upon activating the hand manipulator. In the case of the type of medical instruments mentioned earlier, the swivel handle element of the hand manipulator is designed as a lever, wherein the hinge axis of the two handle elements forms the lever axis. The distance from the hinge axis to the point at which the push/pull rod is located on the handle element is considerably shorter than the distance from the hinge axis to the finger hole on the end of this handle element. The transmission ratio is generally around 10:1, that is, the standard closing force of the hand of about 100 N is amplified tenfold due to mechanical leverage, to around 1,000 N.

When using these medical instruments in practice, in particular the gripping and holding tools, the aim is to generally hold an object, for example a swab or a needle and to place it securely and firmly between the remote tool parts. Strong people can exert a closing force onto the hand manipulator of about 150 N or more, which is then amplified to 1,500 N or more due to mechanical leverage. Frequent use of excess pressure on the remote tool parts can lead to material fatigue or even to the remote tool parts fracturing, whereupon loss of small parts in the operating arena, particularly during an operation can lead to the patient getting injured.

In order to avoid undue excess forces being exerted onto the push/pull rod via the hand manipulator and therefore onto the remote tool parts, a force-limiting device is known in the practical field in which the transmission of force between the hand manipulator and the push/pull forces and/or the remote tool parts is limited by a force-limiting device. This type of force-limiting device is known for example from U.S. Pat. No. 6,589,259 to Solingen. With this known device the push/pull rod is designed as a two-piece component in which both the push/pull rod sections are connected to one another by way of a force-limiting device.

In accordance with another known embodiment configuration the force-limiting device is designed as a spring assembly on the proximal end of the push/pull rod and which absorbs a portion of the force transmitted onto the push/pull rod via the hand manipulator.

These state of the art known force-limiting devices are complicated in construction and are therefore expensive.

Therefore, it is desired to have a method of device removal that precisely grips the cylindrical microstimulator without undue probing while minimizing accompanying tissue damage.

### BRIEF DESCRIPTION OF THE SEVERAL VIEWS OF THE DRAWINGS

FIG. 1 illustrates a side view of a medical device showing the force limiting spring, trigger, and moveable bottom jaw.

FIG. 2 depicts a cross-sectional view of the medical device showing the force limiting spring, inner tube, and activation tube.

FIG. 3 presents a cross-sectional view of the moveable bottom jaw and fixed jaw/inner tube according to an embodiment of the present invention.

FIG. 4 presents a cross-sectional view of the moveable bottom hook and fixed hook.

### DETAILED DESCRIPTION OF THE INVENTION

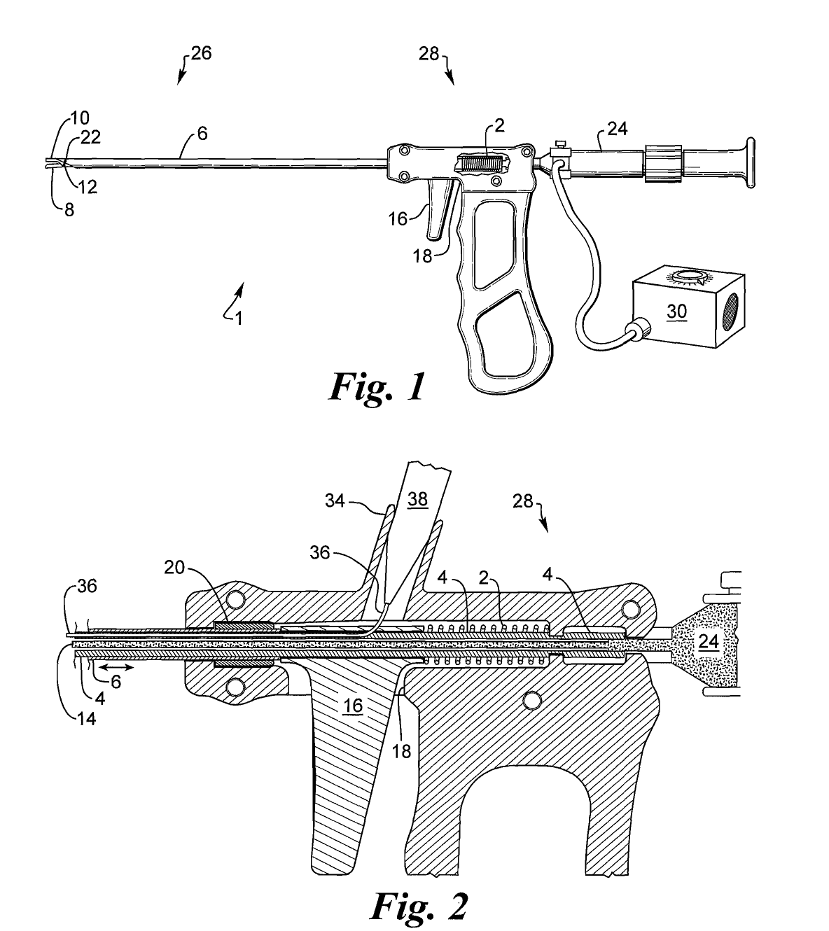

FIG. 1 depicts a medical surgical instrument in the form of a gripping tool 1. The gripping tool 1 has an activation tube 6 which is a hollow cylindrical shaft that extends along its lateral length. On the proximal end 28 of tube 6, a hand manipulator including activation trigger 16 and an optical imaging system 24 with a light source 30 are located.

On the distal end 26 of the activation tube remote tool parts, for example jaws 8, 10 or hooks 42, 44 [FIG. 4], are arranged in the form of two open-ended sections which can be activated via the activation trigger 16 of the gripping tool 1. The trigger 16 moves the activation tube 6 along a line of displacement. The tool includes a fixed jaw 10 and a moveable jaw 8.

Because a hand can exert considerably greater force than 150 N, the gripping tool 1 depicted has a force-limiting spring 2. The force-limiting spring 2 is there to prevent undue excess force being exerted onto the jaws 8, 10 via the activation tube 6 which can lead to damage or even fracturing of the jaws 8, 10.

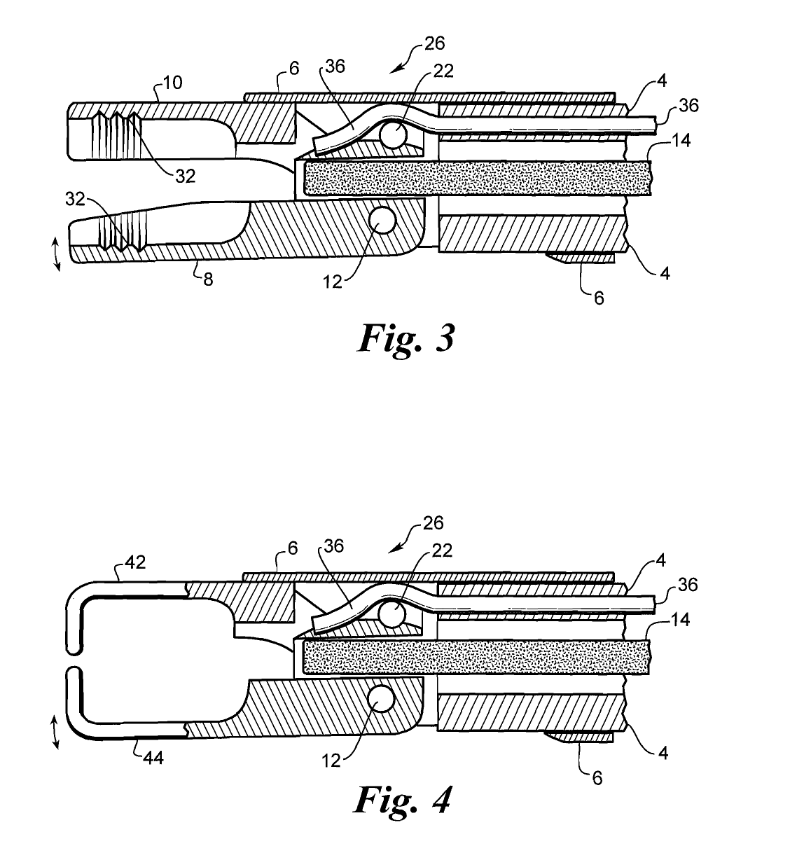

FIG. 2 presents a cross-sectional view of the proximal end 28 showing the activation trigger 16 and the limit stop 18 that limits the trigger 16 movement and therefore prevents the outer activation tube 6 from moving the moveable jaw 8 too closely to fixed jaw 10 during closure, see FIG. 3. The trigger 16 is fixedly attached to activation tube 6, such that when activation trigger 16 is moved toward limit stop 18 by the operator's hand, force-limiting coil spring 2 is compressed by activation tube 6. Activation tube 6 slides laterally along inner tube 4, as presented in FIG. 2. When the closing force is released from trigger 16, force-limiting coil spring 2 urges the activation tube 6 toward the distal end 26, thereby causing movable jaw 8 to move away from fixed jaw 10, thereby opening the jaws and releasing a retained device [not illustrated]. Fixed jaw 10 is preferably immovably attached to inner tube 4 at distal end 26.

The force-limiting coil spring 2 produces linearly increasing force that opposes closure force from activation trigger 16, which could damage an expensive microstimulator implant device. Until trigger 16 contacts limit stop 18, closure force that is transmitted to moveable jaw 8 is limited by force-limiting coil spring 2.

The spring-like elasticity of the coil spring 2 can be adapted and determined by way of the type and number of spring coils as well as the choice of material for the coil spring 2.

Activation tube 6 is guided through a bearing 20, which physically stabilizes the tube 6 position and non-axial movement during operation. It is preferred that bearing 20 be comprised of resinous plastic such as Delrin® from E.I. Du Pont. As activation tube 6 is slidably urged along inner tube 4, the moveable jaw 8 rotates around a fixed pivot 12 by force from a swivel pin 22, which slidably passes through activation tube 6.

The fixed jaw 10 and moveable jaw 8 are preferably shaped to conform to the shape of a cylindrical tube as presented by a known microstimulator, such as a Bion having a 6 mm diameter. This curvature minimizes or prevents contact damage during the gripping operation due to stress concentration at the contact point between the microstimulator and the jaws 8, 10. Further, in a preferred embodiment, the gripping surface of the jaws 8, 10 are provided with a roughened or knurled surface 32 to provide a non-slip surface for contact with the microstimulator being gripped.

In an alternate embodiment, an optical imaging system is included. This may be a fiberscope, which comprises an eyepiece at one end and a lens at the other and which may contain a light source, including fiber optics 14 to carry light from the light source 30 at the proximal end 28 to the distal end 26 to provide illumination at the distal end 26 during a gripping operation. The fiber optics 14 pass along the central axis of the inner tube 4 and provide an optical view at the distal end 26 of gripping tool 1. The fiber optics 14 also pass through the movable jaw 8, as illustrated in FIG. 3, and the fiber optics 14 are bent slightly as the movable jaw 8 produces an opening and closing motion.

The force-limiting spring 2 depicted in FIGS. 1 and 2 is distinguished by being simple and cost effective to manufacture without additional components.

In another alternative embodiment as depicted in FIGS. 2 and 3, a port 34 is integral with the gripping tool 1 and is attached to conical fitting 38 to supply saline via tube 36 for a saline flush of the implant location, which passes along gripping tool 1 to the distal end 26, as presented in FIG. 3. Tube 36 releases the saline at the distal end 26 of activation tube 6. The tube 36 can also be used to drain biofluid, for example, from the distal end 26.

An alternative embodiment, FIG. 4, utilizes a fixed hook 42 and a moveable hook 44 to capture an implanted device by its eyelet. If the microstimulator has an eyelet, this lends itself to removal by the hooks presented and further reduces damage to the living tissue or the implant itself.

Obviously, many modifications and variations of the present invention are possible in light of the above teachings. It is therefore to be understood that, within the scope of the appended claims, the invention may be practiced otherwise than as specifically described.

The invention claimed is:

1. A medical instrument comprising:
remote tool parts on a distal end of the medical instrument for grasping an implanted device for removal;
an activation tube displaceable along a line of displacement by an activation trigger arranged on a proximal end of said activation tube adapted to activate said remote tool parts at the distal end of said activation tube;
a force-limiting device comprising a coil spring coaxially arranged with said activation tube for limiting the transmission of force to the remote tool parts from said activation trigger.

2. The medical instrument according to claim 1, further comprising an optical imaging system comprised of lens, fiber optics and a light source.

3. The medical instrument according to claim 2, wherein said imaging system is arranged in a collinear orientation with said activation tube.

4. The medical instrument according to claim 1, wherein said remote tool parts are comprised of cylindrically shaped jaws to facilitate grasping an implanted device for removal.

5. The medical instrument according to claim 4, wherein said cylindrically shaped jaws have a radius of about 6 mm.

6. The medical instrument according to claim 1, wherein said remote tool parts are comprised of a fixed jaw and a jaw that is moveable by said activation tube.

7. The medical instrument according to claim 6, wherein said activation tube surrounds a fixed inner tube that is arranged collinearly with said activation tube and that is integral with said fixed jaw.

8. The medical instrument according to claim 1, wherein the spring-like elasticity of said coil spring can be adjusted through its shape and/or the number of the coils.

9. The medical instrument according to claim 1, wherein the elasticity of said coil spring can be adjusted via the material comprising said coil spring.

10. The medical instrument according to claim 1, wherein said activation tube and said coil spring are independently arranged.

11. The medical instrument according to claim 1, wherein said coil spring has a virtually constant cross section.

12. The medical instrument according to claim 1, further comprising a port and tube for saline flushing.

13. The medical instrument according to claim 1, wherein said remote tool parts are comprised of a fixed hook and a hook that is moveable by said activation tube.
